(19) United States
(12) Patent Application Publication
(10) Pub. No.: US 2018/0360415 A1
(43) Pub. Date: Dec. 20, 2018

(54) Ultrasound Gel Container

(71) Applicant: HR Pharmaceuticals, Inc., York, PA (US)

(72) Inventor: Ryan Dohm, York, PA (US)

(73) Assignee: HR Pharmaceuticals, Inc., York, PA (US)

(21) Appl. No.: 15/624,096

(22) Filed: Jun. 15, 2017

(51) Int. Cl. A61B 8/00

(52) U.S. Cl. CPC A61B 8/4281

(57) ABSTRACT

An ultrasound coupling gel container is provided and includes a flexible body having front wall, back wall, an outlet end, a planar bottom gusset, and a dispensing assembly. The back wall is positioned adjacent and opposes the front wall. The back wall is sealed to the front wall along a periphery thereof and provides a cavity receiving section. An outlet end is formed from a front wall top section and a back wall bottom section. The planar bottom gusset includes a central portion formed from sealed peripheral edge portions along the front wall and of the back wall. The dispensing assembly is sealed to a peripheral edge of the front wall and of the back wall at the outlet end of the flexible body.

### FIELD OF THE INVENTION

This invention relates to a flexible container and, more particularly, to a flexible, collapsible container for dispensing an ultrasound coupling gel composition.

### BACKGROUND

Ultrasound diagnostic procedures are used in assessing and diagnosing a wide variety of medical conditions related to internal organs. These procedures are widely used in obstetrics and cardiology, among other medical specialties.

An ultrasound coupling agent, otherwise known as a “gel”, is designed to act as a conductive barrier between an ultrasound probe and the outer layer of skin, known as the epidermis. The application of a gel to the patient's body lubricates the skin and prevents the sound waves from being trapped or reflected by air pockets that might distort the image and lead to an incorrect diagnosis.

Known ultrasound coupling gel containers have disadvantageous features that interfere with storage, transport, and the optimal application of coupling gel. Flexible containers with dispensing ports are known. U.S. Pat. No. 6,050,451 (Hess et al.) describes a dispensing structure adaptable for use with thin-walled, flexible, containers used for beverages.

### SUMMARY

An ultrasound coupling gel container is provided and includes a flexible body having front wall, back wall, an outlet end, a planar bottom gusset, and a dispensing assembly. The back wall is positioned adjacent and opposes the front wall. The back wall is sealed to the front wall along a periphery thereof and provides a cavity receiving section. An outlet end is formed from a front wall top section and a back wall bottom section. The planar bottom gusset includes a central portion formed from sealed peripheral edge portions along the front wall and of the back wall. The dispensing assembly is sealed to a peripheral edge of the front wall and of the back wall at the outlet end of the flexible body.

Improved ultrasound gel container products are desirable for better container transport, storage, and use in dispensing ultrasound gel product. A compact and easily graspable shape is desirable in order to facilitate the handling and storage of full and empty gel containers. An improved gel container would have features that allowed for convenient storage and display. The container would advantageously have a dispensing assembly sealed to the container. The dispensing assembly used in the gel container would maintain a desirable gullet at the entrance to the dispensing cap to permit the optimal dispersal of ultrasound gel. The body of the gel container would be manufactured of a polymeric multi-layered material to provide desirable characteristics of flexibility and printability. The gel container holds a volume of gel that is sufficient and easily dispersed by the operator for a particular procedure or treatment, preferably in an amount sufficient for a single procedure that prevents microbial contamination.

### BRIEF DESCRIPTION OF THE DRAWINGS

These and other features, aspects, and advantages of the invention will become better understood with regard to the following description, appended claims, and accompanying drawings which form a part of the specification and in which like numerals are used to identify like parts throughout and where:

FIG. 1 is a perspective view of a gel container in use;

FIG. 2 is another perspective view of the gel container in FIG. 1, showing a user dispensing a gel stored therein;

FIG. 3 is a plan view of a gel container body according to the invention, shown before assembly;

FIG. 4 is a perspective view of a blank sheet of gel containers according to the invention, before assembly thereof;

FIG. 5 is a perspective exploded view of a gel container according to the invention, shown prior to assembly.

FIG. 6 is a front view of a gel container according to the invention;

FIG. 7 is a left side view of the gel container of FIG. 6;

FIG. 8 is a front view of the gel container of FIG. 6, shown filled with a gel;

FIG. 9 is left side view of the gel container of FIG. 8;

FIG. 10 is a partial front, plan view of a base of a gel container according to the invention;

FIG. 11 is a sectional view of the gel container of FIG. 10 along line 11-11.

FIG. 12 is a sectional view of the gel container of FIG. 10 along line 12-12.

FIG. 13 is a sectional view of the gel container of FIG. 10 along line 13-13.

FIG. 14 is a bottom view of a gel container according to the invention, shown filled with a gel;

FIG. 15 is a perspective view of the gel container of FIG. 14;

FIG. 16 is a front view of a base end of another gel container according to the invention;

FIG. 17 is a perspective view of the gel container of FIG. 16;

FIG. 18 is a bottom view of the gel container of FIG. 16; and

FIG. 19 is a perspective view of the gel container of FIG. 16.

### DETAILED DESCRIPTION OF THE EMBODIMENT(S)

The following text sets forth a broad description of various exemplary embodiments of the invention. The description is to be construed as exemplary only and does not describe every possible embodiment since describing every possible embodiment would be impractical, if not impossible. It will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims. All publications and patents cited herein are incorporated herein by reference.

Unless defined otherwise, all technical and scientific terms used herein have the same meaning as commonly understood by one of ordinary skill in the art to which this invention belongs. In case of conflict, the present application including the definitions will control. In addition, unless otherwise required by context, singular terms shall include pluralities and plural terms shall include the singular. All publications, patents, and other references mentioned herein are incorporated by reference in their entireties for all purposes.

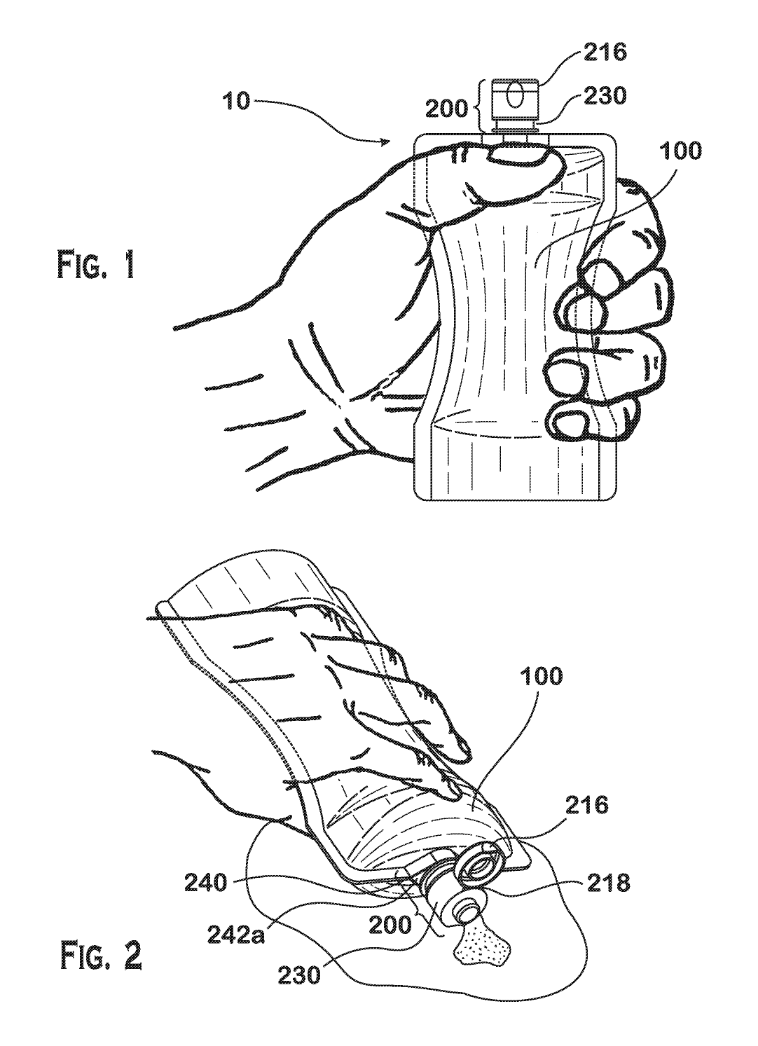

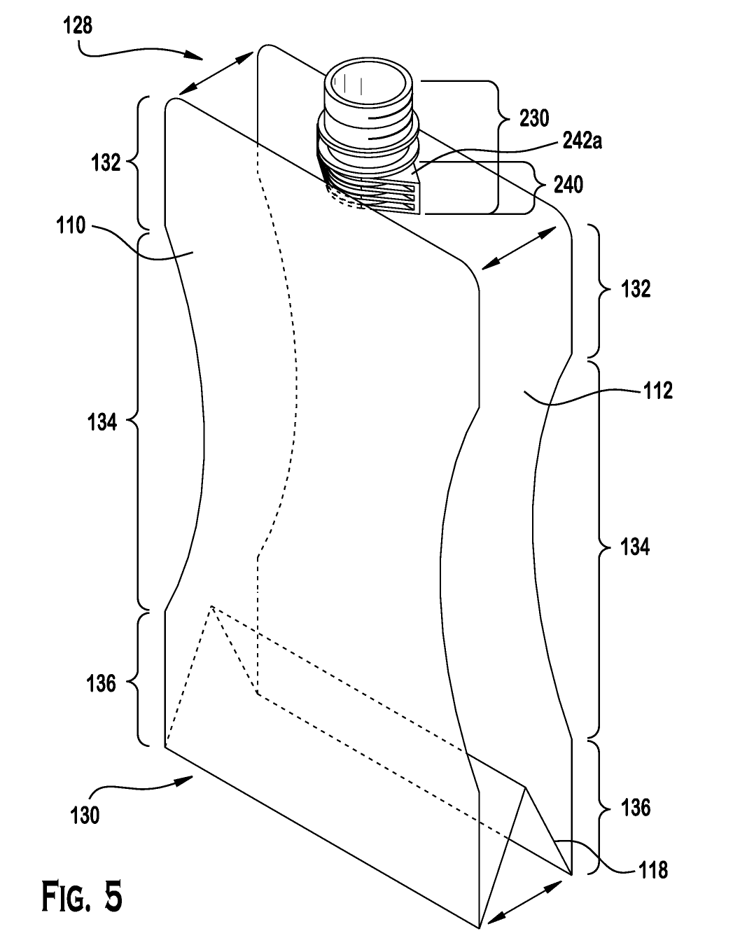

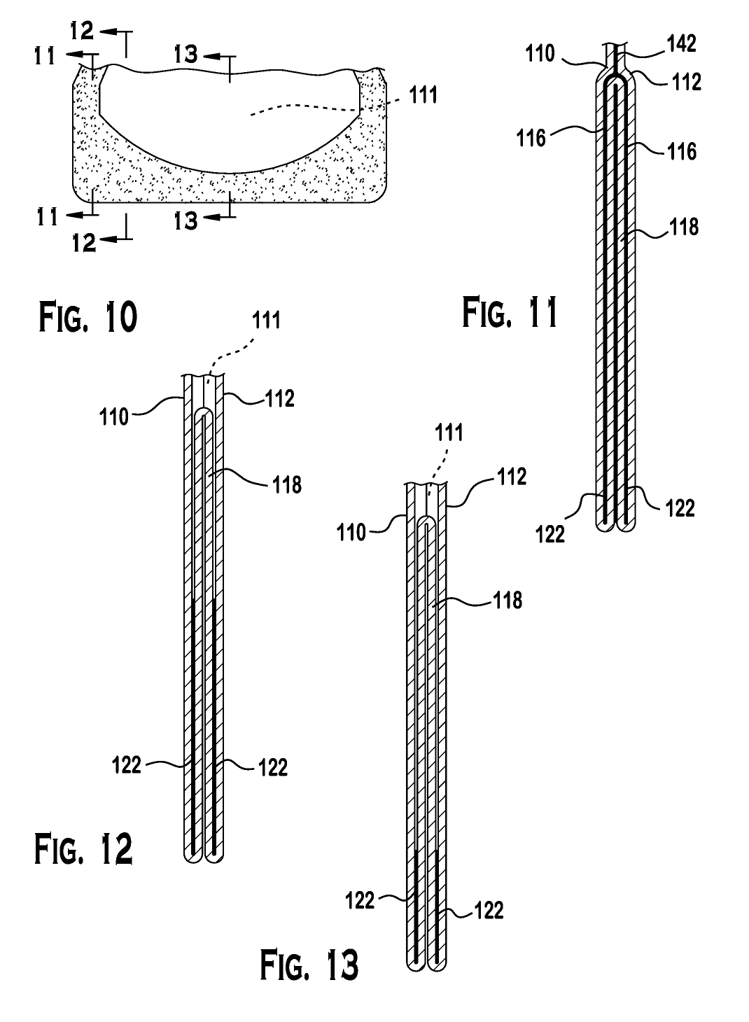

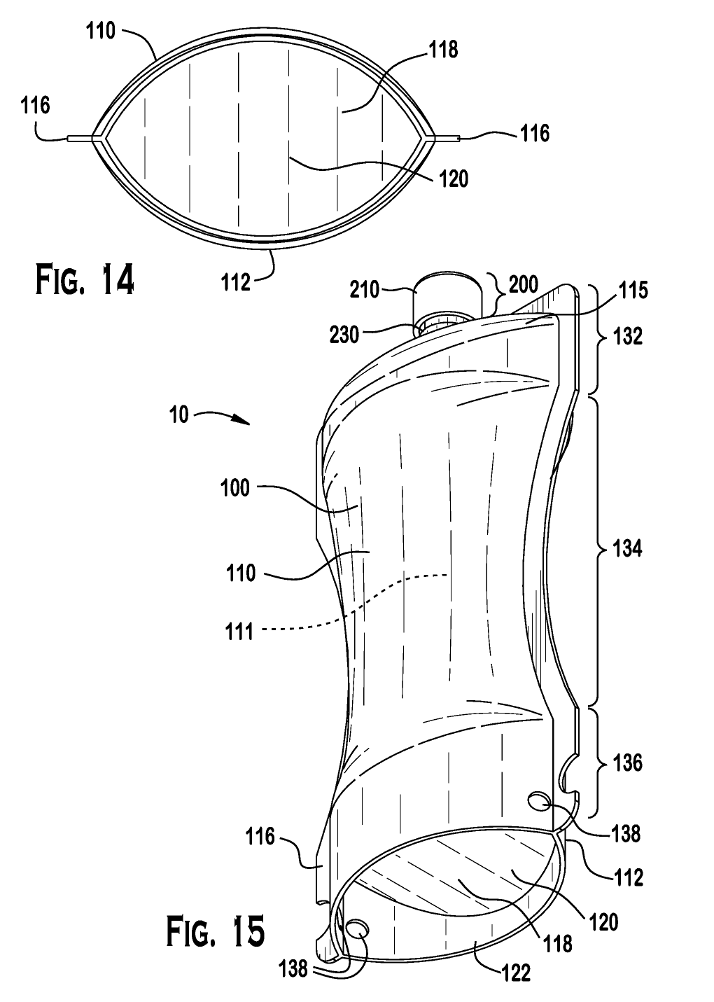

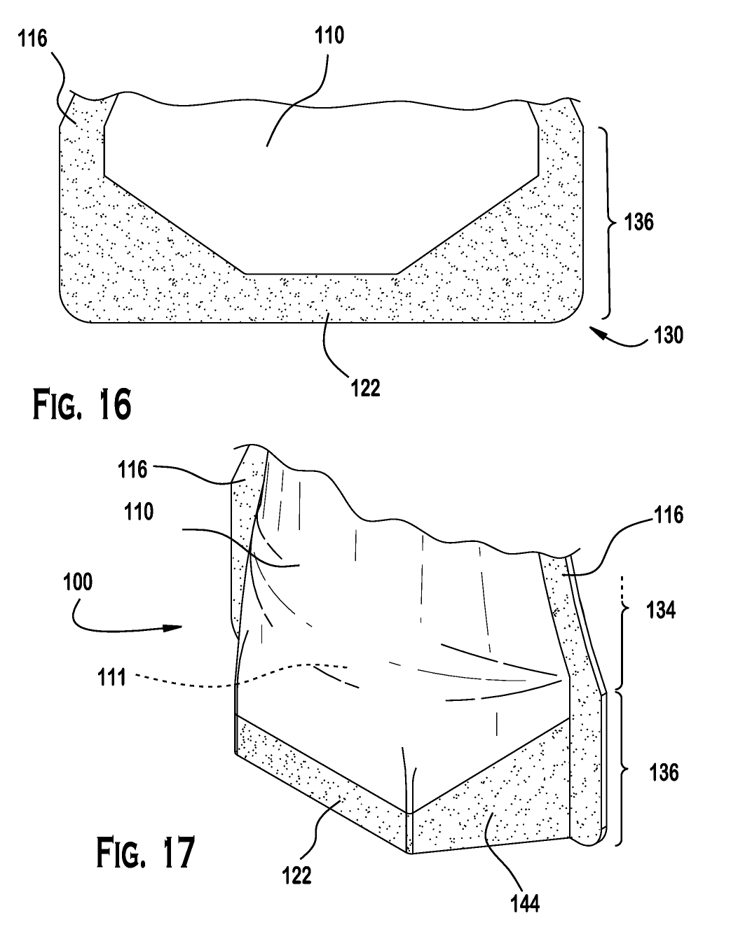

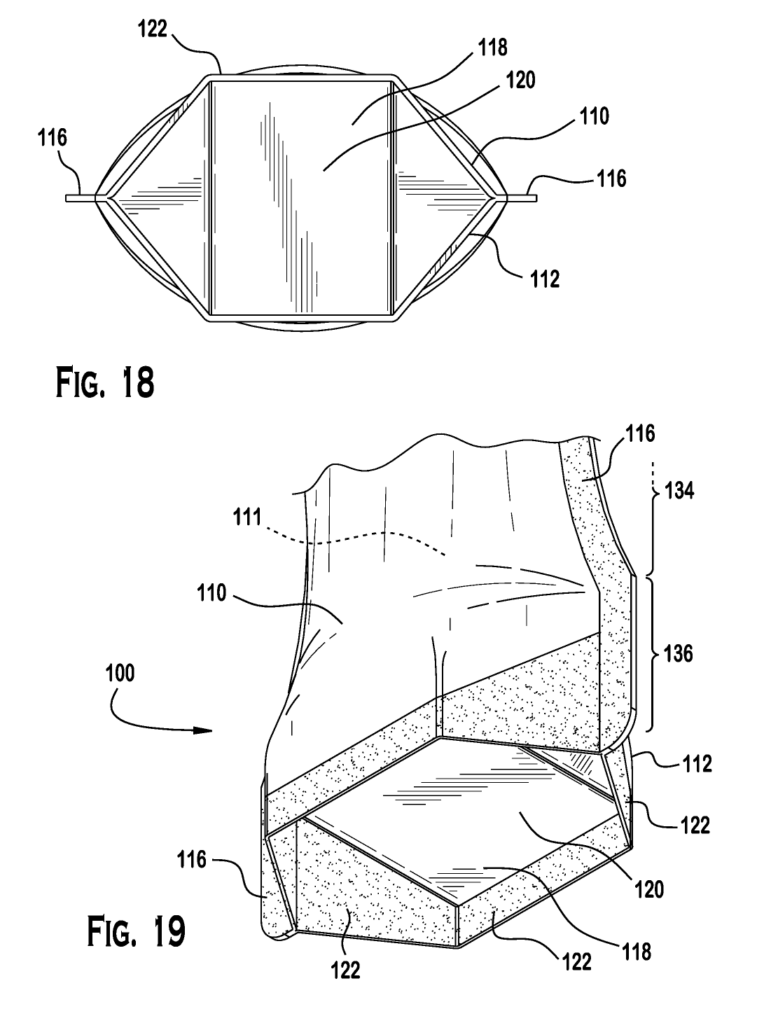

Now, with reference to FIGS. 1-19, an ultrasound coupling gel the container 10 (hereinafter referred to as “the container 10”) according to the invention is shown. An exemplary embodiment of the container 10 includes the following major components: a body 100 and a dispensing assembly 200.

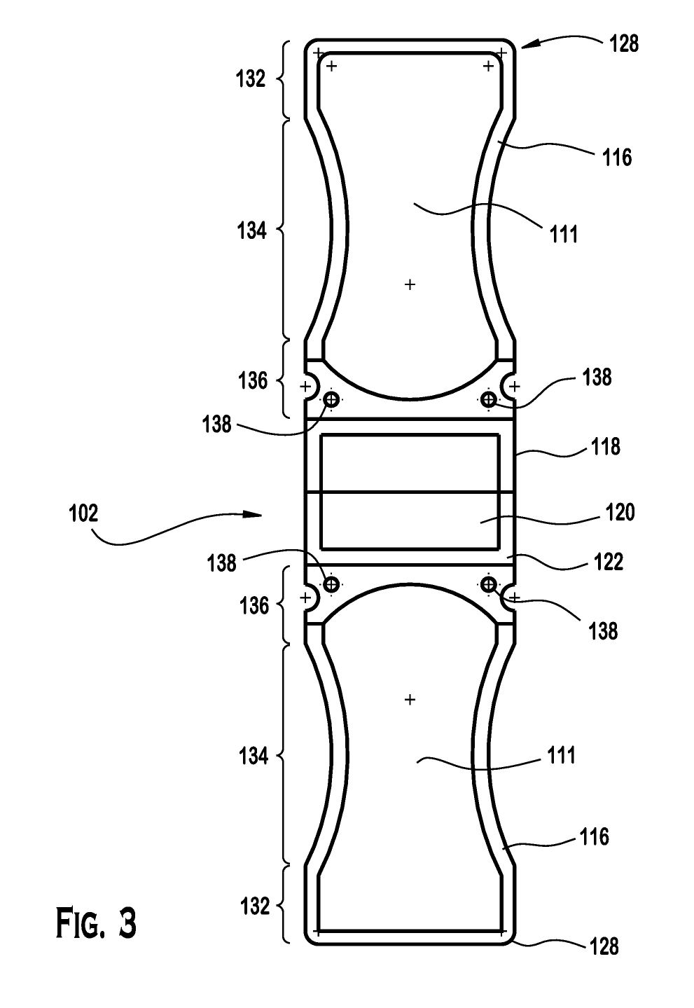

As shown, the body 100 is a substantially planar, enclosed flexible container having a front wall 110, a back wall 112, and a bottom gusset 118. The front wall 110 and the back wall 112 are positioned so that they oppose and mirror each other. The body 100 extends between an outlet end 128 and a base end 130. The outlet end 128 is considered an upper end of the container 10, while the base end 130 is considered as a lower end of the container 10. This is not the only operable orientation and the container 10. Rather, the container can be used in any position, such as inverted, on its side, or the like. FIGS. 1 and 2 show two options for handling and use of the container 10.

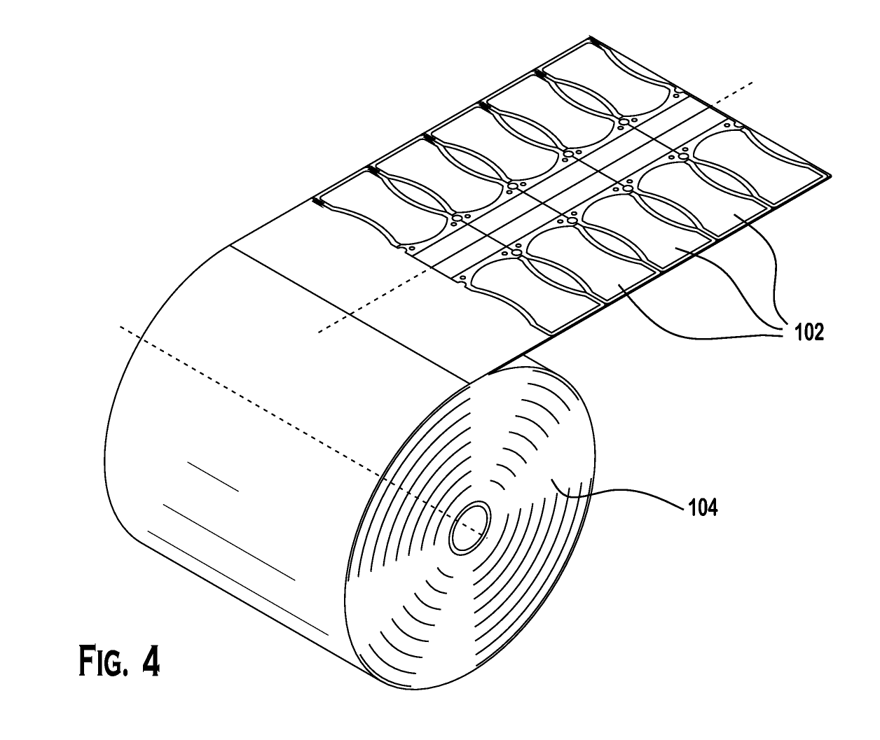

FIG. 3 shows a single blank 102 from which the body 100 is constructed. FIG. 4 shows a continuous length of polymeric material 104 on which multiple blanks 102 are fitted for construction of individual bodies 100.

As shown in FIGS. 1-6, 8-9, and 15, the width of the body 100, along a lateral axis, is less at a mid-point on along a longitudinal axis 124 than at the outlet end 128 and at the base end 130. As a result, the body has an arcuate shape in an exemplary embodiment of the invention. The blank 102 includes a shoulder portion 132 along an outlet end 128, an arcuate-shaped waist portion 134, and a hip portion 136 along the base end 130. The hip portion 136 is a section of the front wall 110 and the back wall 112. The front wall 110 and the back wall 112 are sealed to each other along a peripheral edge 116 to form a cavity receiving section 111. Curvatures of front wall 110 and back wall 112 and the curvatures of the side peripheral edges 116 have certain orientations relative to planes passing through the center of symmetrical body of the container 10. When constructed, the container's shape is sized such that it may be griped along the waist portion 134 by a hand, in order to permit convenient single-handed dispensing of a gel by the user.

The construction of the base end 130 allows for easy storage and handling of both filled and unfilled gel containers 10, and will now be described.

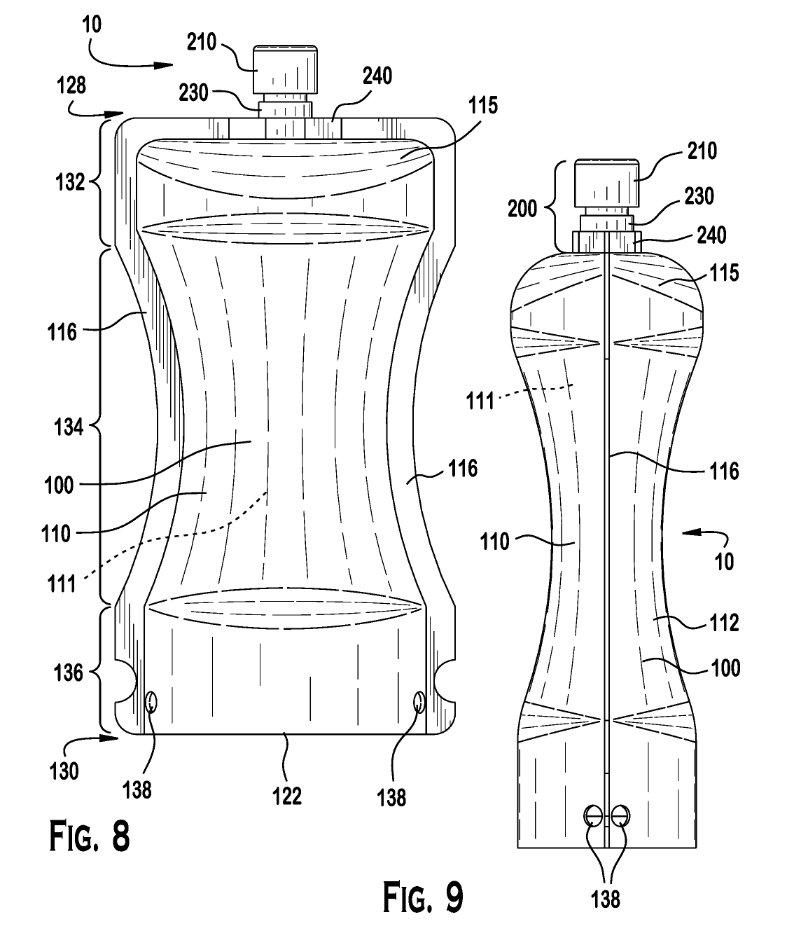

In an exemplary embodiment of the invention, the planar bottom gusset 118 is substantially planar and rectangular. The planar bottom gusset 118 includes a central portion 120 and peripheral edge portions 122. In the shown embodiment, the planar bottom gusset 118 is sonically or heat sealed along the peripheral edge portions 122. As shown, two opposing peripheral edges 122 are sealed to peripheral edges 116 of lower front wall 110 and of lower back wall 112 along the base end 130 thereof. The central portion 120 of bottom gusset 118 remains unsealed. A portion of the remaining two opposing peripheral edges 122 of bottom gusset 118 are respectively laminated between and sealed to the lower peripheral side edges 116 of front wall 110 and of back wall 112. This arrangement of sealing of the peripheral edges is especially shown in FIGS. 10-13 and 17-19.

Sealing of the planar bottom gusset 118 to the front wall 110 and the back wall 112 is performed such that the unsealed central portion 120 lies generally flat and perpendicular to the longitudinal plane of body 100 as the container 10 is filled. This design configuration enables the container 10, when filled, to be positioned upright along its base end 130.

Additionally, this configuration enables the container 10, when empty, to be folded so that the planar bottom gusset 118 will lie flat along a common plane with the front wall 110 and the back wall 112. In an exemplary embodiment of the invention, the container 10 has a substantially elliptical cross-sectional shape when filled, as shown in FIG. 15. The sealed peripheral side edges 116 are positioned parallel to a major longitudinal axis 124 of the ellipse. However, when the container 10 is not filled, the front wall 110 and the back wall 112 are positioned adjacent, and oppose each other, along a common plane.

In an exemplary embodiment and as shown in FIGS. 6 and 7, the outlet end 128 is three-dimensional structure, formed by the front wall 110 with a first inset gullet 140a and back wall 112 with a second inset gullet 140b positioned about the longitudinal central line 124, respectively. This three-dimensional structure is designed to accommodate the dispensing assembly 200, which is then surrounded by and sealed to the body 100, as described below. The first inset gullet 140a and second inset gullet 140b are triangular and planar and are sized to be fitted and sealed within notches cut in front wall 110 and back wall 112 using sonic or heating methods known to one of ordinary skill in the art. More specifically, upper edges of first inset gullet 140a and of second inset gullet 140b are sufficiently longer than the notch formed along the upper edge of front wall 110 and back wall 112. This design permits a collar 240 of the dispensing assembly 200, as described below, to be sealed along the outlet end 128. The first inset gullet 140a and second inset gullet 140b maintain an unobstructed dispensing point 126 for the application of a gel through the dispensing assembly.

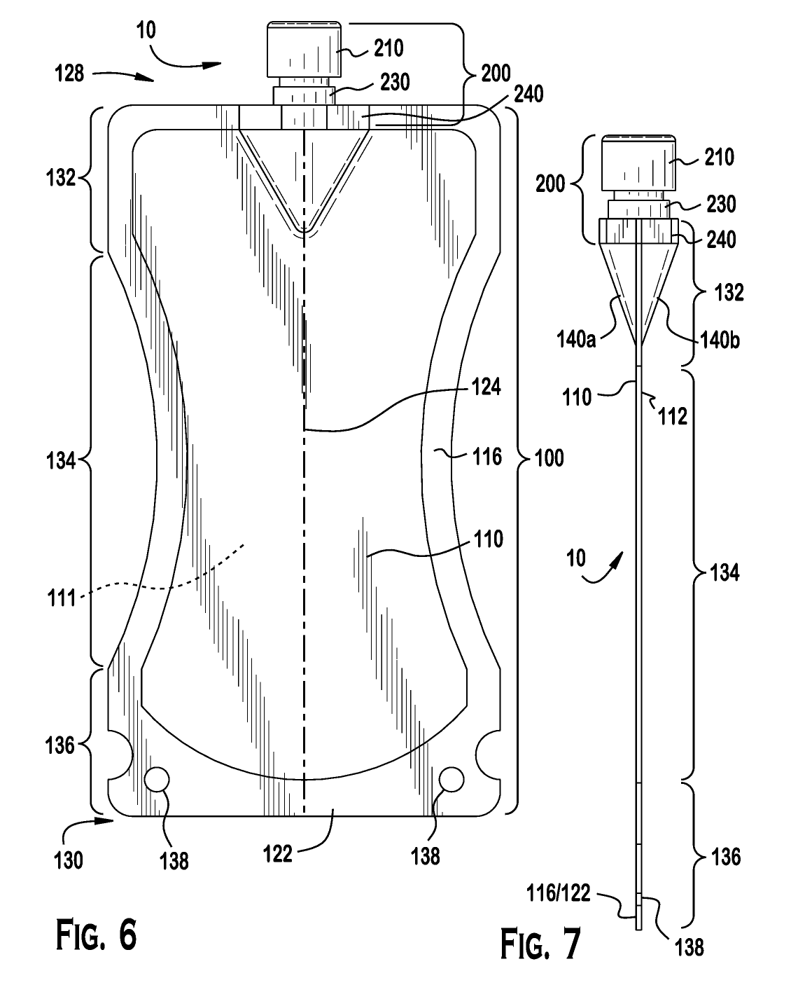

In yet another embodiment, the front wall 110 and back wall 112 include pleats 115 as shown in FIGS. 8-9 and 15. During filling, the pleats unfold to accommodate increasing gel volume.

Additionally, as seen in FIGS. 3, 6, 8, 9, and 15, the body 100 includes receiving passageways 138 along the sealed peripheral edges of base end 130. These receiving passageways 138 are useful for receiving a hanging device, such as fastener, hook, clamp, or other known devices for suspending products during storage or use. Exemplary receiving passageways 138 include, but are not limited to, an eye, a hole, a slit, a hook, a loop, a grommet, a clamp, or other feature to suspend the container 10.

The dimensions of the container 10, including the width of the sealed peripheral edges 116, 122, are chosen relative to a desired container volume and to permit the container 10 to rest upright on sealed peripheral edges of base end 130 when filled or at least partially filled with gel.

The outlet end 128 of body 100 is proportioned to accommodate the sealing of a dispensing assembly 200 as described below. As illustrated in FIGS. 1-2, 5-9, and 15, the container 10 additionally includes a dispensing assembly 200 hermetically sealed to the outlet end 128. The dispensing assembly 200 provides a sealable closure for the container 10. The dispensing assembly 200 is selected by one of ordinary skill in the art of packaging in light of the properties of the particular gel to be contained and the gel's application conditions. Dispensing assembly 200 is positioned between first inset gullet 140a of front wall 110 and second inset gullet 140b of back wall 112 along the outlet end 128 of the container 10. The dispensing assembly 200 is positioned along the perimeter edges of the container 10. The dispensing assembly 200 may be sonically or heating sealed to the container. One skilled in the art should appreciate that the other known methods to seal the dispensing assembly may be used.

In a further embodiment of the invention, the dispensing assembly 200 may additionally include a tamper-revealing purity seal (not shown) applied at the upper edge of nozzle 230. The tamper-revealing purity seal is removed by the user before dispensing gel from the container 10.

The dispensing assembly 200 may include a snap or flip cap, a threaded screw cap, a peel off seal, or a stopper, and may be resealable. The dispensing assembly 200 preferably includes a threaded screw cap 210 or a flip cap 216. An embodiment of the dispensing assembly 200 using a flip cap 216 is shown in FIGS. 1 and 2. An embodiment of the dispensing assembly 200 using a threaded screw cap 210 is shown in FIGS. 6-9 and 15.

Threaded screw cap 210 or flip cap 216 are preferably unitary and formed of a semi-rigid plastic that fits to a nozzle 230. Nozzle 230 is preferably unitary and formed of a semi-rigid plastic in a tube configuration to mate or connect effectively with a cap or other resealing mechanism. The nozzle 230 has a cylindrical receiving passageway and is formed by injection molding from plastic or by other suitable methods and materials known to those of ordinary skill in the art. The nozzle 230 is adapted to connect to a flip cap 216 or threaded screw cap 210. In another embodiment, the flip cap 216 may be connected to the nozzle 230 with a hinging element 218.

As shown in FIGS. 1-2, 5-9, and 15, nozzle 230 has a collar 240 at its lower end. The collar 240 may be described as “boat-shaped” with a first flange 242a and a second flange 242b extending opposite each other. When in position, first flange 242a and second flange 242b are positioned in a plane generally perpendicular to the longitudinal central line 124 through the center axis of nozzle 230. The outside surface of first flange 242a and second flange 242b are sealed within the peripheral edge of first inset gullet 140a and second inset gullet 140b of front wall 110 and back wall 112, respectively. Flanges 242a, b may optionally have a ribbing 244 to improve sealing to first and second inset gullets 140a, b. The vertical heights of first flange 242a and of second flange 242b are uniform and correspond to the width of the peripheral edge 116 of first inset gullet 140a and second inset gullet 140b. One suitable choice for a collar 240 may be found in U.S. Pat. No. 6,050,451.

The front wall 110, back wall 112, and bottom gusset 118 are preferably made of the same material. Preferably, the walls of the container 10 are formed of a flexible packaging laminate material having a thickness that is uniform or at least uniformly symmetrical about the longitudinal central line 124 so that the walls 110, 112, the outlet end 128, and the base end 130 will develop stresses uniformly.

According to the invention, the polymeric material 104 is a multi-layered flexible polymeric material, such as Winpack WCS100, produced by and available from Winpack (21919 Dumberry Road, Vaudreuil-Dorion, Quebec, Canada J7V 8P7, www.winpack.com). The multi-layered flexible polymeric material allows for hermetic sealing and permits easy filling. In one exemplary embodiment, the multi-layered flexible polymeric material includes a coextruded layer, a tempered foil layer, a low density polyethylene (LDPE) layer, and a polyethylene terephthalate (PET) layer. In an exemplary embodiment of the invention, the coextruded layer is a coextruded layer of more than one polymers and, more particularly, an 18 lb/ream coextruded layer of more than one polymers. In an exemplary embodiment of the invention, tempered foil is about 0.000285″ thick. In an exemplary embodiment of the invention, the LDPE layer is a 7.0 lb/ream white sheet. In an exemplary embodiment of the invention, the PET layer is a 48 ga sheet of material. In an exemplary embodiment of the invention, the multi-layered flexible polymeric material has a total thickness of 2.40 mil+/−20%. Similar polymeric materials available from the same manufacturer can be printed using reverse rotogravure and surface flexography. Other similar polymeric flexible packaging laminates are known to those of ordinary skill in the art

Now, use of the container 10 according to the invention will be described. In operation and as illustrated in FIGS. 1-2, the user removes the threaded screw cap, flip cap, or other cap, removes the tamper-revealing purity seal, if any, and positions the gel the container 10 so as not to obstruct the dispersal of gel. The user exerts pressure, typically with one hand, on the front wall 110 and back wall 112 of the container 10 at its waist portion 134 to create an inwardly directed pressure. In response to this pressure, gel is dispensed out of the dispensing assembly 200 to a location as directed by the user that is appropriate for the diagnostic or treatment ultrasound procedure. The contents of the container 10 are forced outwardly through nozzle 230 by the dispensing pressure. The user then discontinues the dispensing pressure inward on front wall 110 and back wall 112. The dispensing cycle is repeated as indicated by the procedure.

In an exemplary embodiment of the invention, as the container 10 is emptied of gel, the opposing front wall 110 and back wall 112 are urged inward, towards each other. This can be seen by comparing FIGS. 1, 2, 9, 14-15, and 17-19 when filled with FIGS. 6-7 and 10-13 when unfilled and collapsed. A fully emptied container 10 may be folded as described above for convenient disposal.

The container 10 according to the invention is preferably disposable. It is preferably designed for one time use to reduce opportunities for microbial contamination of equipment and patients. The volume of the container is chosen relative to the ultrasound procedure undertaken and preferably ranges from 0.5 oz. to 600 oz.

The claimed invention also includes a kit including at least one gel container containing ultrasound coupling gel.

The foregoing illustrates some of the possibilities for practicing the invention. Many other embodiments are possible within the scope and spirit of the invention. It is, therefore, intended that the foregoing description be regarded as illustrative rather than limiting, and that the scope of the invention is given by the appended claims together with their full range of equivalents.

What is claimed is:

1. An ultrasound coupling gel container, comprising
a flexible body having:
a front wall;
a back wall positioned adjacent and opposing the front wall, the back wall sealed to the front wall along a periphery thereof and providing a cavity receiving section;
an outlet end formed from a front wall top section and a back wall bottom section, the outlet end a planar bottom gusset having a central portion formed from sealed peripheral edge portions along the front wall and the back wall;
a dispensing assembly sealed to a peripheral edge of the front wall and of the back wall along the outlet end.

2. The ultrasound coupling gel container according to claim 1, wherein a width of the flexible body is less about at a longitudinal mid-point thereof than a width of the outlet end and the planar bottom gusset.

3. The ultrasound coupling gel container according to claim 1, wherein the front wall and the back wall include an arcuate edge.

4. The ultrasound coupling gel container according to claim 1, wherein the front wall includes an inset gullet positioned along the outlet end and secured to the dispensing assembly.

5. The ultrasound coupling gel container according to claim 4, wherein the back wall each further comprise an inset gullet at the outlet end and sealed to the dispensing assembly.

6. The ultrasound coupling gel container according to claim 1, wherein the dispensing assembly includes a closure.

7. The ultrasound coupling gel container according to claim 6, wherein the closure is a snap-off closure.

8. The ultrasound coupling gel container according to claim 1, wherein the flexible body is a continuous sheet of a multi-layered flexible polymeric material.

9. The ultrasound coupling gel container according to claim 8, wherein the planar bottom gusset is formed from a folded and laminated bottom section of the front wall and a folded and laminated top section of the bottom wall.

10. The ultrasound coupling gel container according to claim 9, wherein the multi-layered flexible polymeric material includes a composition having a tempered foil layer, a low-density polyethylene (LDPE) layer, and a polyethylene terephthalate (PET) layer.

11. The ultrasound coupling gel container according to claim 10, wherein a thickness of the multi-layered flexible polymeric material has a total thickness of about 2.40 mil.

12. The ultrasound coupling gel container according to claim 1, wherein the flexible body includes a receiving passageways extending through one of the sealed peripheral edge portions.

13. The ultrasound coupling gel container according to claim 1, further comprising an ultrasound coupling gel positioned in the cavity receiving section.

14. The ultrasound coupling gel container according to claim 13, wherein the planar bottom gusset can expands outwardly as the ultrasound coupling gel is positioned in the cavity receiving section.

15. The ultrasound coupling gel container according to claim 14, wherein the front wall and back wall are urged toward each other when the ultrasound coupling gel is positioned in the cavity receiving section.
